(19) United States
(12) Patent Application Publication
(10) Pub. No.: US 2011/0165138 A1
(43) Pub. Date: Jul. 7, 2011

(54) PROTOCOLS FOR TREATING AND PREVENTING OBESITY AND COMPLICATIONS ARISING THEREFROM

(75) Inventors: Antonio Rajic, West Preston Victoria (AU); Dominic J. Autelitano, Camberwell Victoria (AU); Ana K. Vrkic, Bundoora Victoria (AU); Christopher G. Hosking, Nunawading Victoria (AU); Leodevico L. Ilag, Balwyn Victoria (AU)

(73) Assignee: HEALTHLINX LIMITED, RICHMOND VICTORIA (AU)

(21) Appl. No.: 13/000,104

(22) PCT Filed: Jun. 17, 2009
(86) PCT No.: PCT/AU2009/000776

§ 371 Date: Mar. 9, 2011

(30) Foreign Application Priority Data
Jun. 26, 2008 (AU) 2008903257

(51) Int. Cl. A61K 38/17; A61K 38/44; C07K 14/47; A61P 3/04; A61P 9/00; A61P 3/10; A61P 3/00; C12N 9/08; C12Q 1/02

(52) U.S. Cl. 424/94.4; 514/4.8; 530/395; 530/350; 435/192; 435/29

(57) ABSTRACT

The present invention relates generally to the field of obesity and complications arising therefrom and in particular to the control of obesity by inhibiting adipogenesis and related processes.

This application is associated with and claims priority from Australian Patent Application No. 2008903257, filed on 26 Jun. 2008, the entire contents of which, are incorporated herein by reference.

### FIELD

The present invention relates generally to the field of obesity and complications arising therefrom and in particular to the control of obesity by inhibiting adipogenesis and related processes.

### BACKGROUND

Bibliographic details of the publications referred to by author in this specification are collected alphabetically at the end of the description.

Obesity has become an epidemic problem in Western societies contributing to several disease processes including metabolic diseases, hypertension, and cardiovascular disease. Adipogenesis is defined as the formation of fat or fatty tissue, the development of fat cells from pre-adipocytes, has been one of the most intensely studied models of cellular differentiation. In part this has been due to the availability of in vitro models which recapitulate most of the critical aspects of fat cell formation in vivo. The mouse 3T3-L1 preadipocyte cell line is one of the best characterised models of adipocyte differentiation. In vitro, maximal differentiation into mature adipocytes is achieved by treating post-confluent cells with a cocktail containing fetal bovine serum, insulin, the glucocorticoid agonist dexamethasone and an agent such as IBMX that elevates cAMP (Student et al, J. Biol. Sci. 255(10):4745-4750, 1980). A time-dependent sequence of events occurs that triggers the activation of a distinct set of transcription factors and down-stream target genes that ultimately gives rise to the mature adipocyte phenotype and the capacity to dramatically increase de novo synthesis and storage of lipids (Smas and Sul, Biochem. J. 309:697-710, 1995; Morrison and Framer, J. Nutrit. 130(12):3116S-3121S, 2000; Ntambi and Kim, J. Nutrit. 130(12):3122S-3126S, 2000). In the early stages of differentiation, induced preadipocytes undergo a post-confluent mitosis at about 24 hours and subsequent growth arrest that commits the cell to differentiation to an adipocyte phenotype. Alterations in the regulation and expression of as many as 300 proteins may be associated with the process of preadipocyte differentiation. In particular, very early events following induction of the preadipocyte involve transcriptional activation of family members of the CCAAT/enhancer-binding proteins such as C/EBPβ and C/EBPγ which in turn activate expression of the peroxisome proliferator-activated receptor, PPARγ (Smas and Sul, 1995 supra; Morrison and Framer, 2000 supra; Ntambi and Kim, 2000 supra). Activation of sterol regulatory binding proteins (SREBPs) and repression of AP-2a and SP1 transcriptional activity are also considered to be crucial early events in adipocyte differentiation (Smas and Sul, 1995 supra; Morrison and Framer, 2000 supra). The expression of PPARγ and C/EBPα in particular are considered to be crucial in regulating genes involved in the maturation of the adipocyte phenotype (Smas and Sul, 1995 supra; Morrison and Framer, 2000 supra; Ntambi and Kim, 2000 supra). Late markers of differentiation including adipocyte specific enzymes involved in lipid metabolism begin to be expressed by day 3 and terminally differentiated adipocytes containing obvious cytoplasmic lipid droplets are evident by days 5-7 (Smas and Sul, 1995 supra; Ntambi and Kim, 2000 supra).

The hormonal induction of preadipocyte differentiation can be mediated by a variety of factors including insulin which acts via the insulin-like growth factor-1 (IGF-1) receptor, corticosteroids acting via the glucocorticoid receptor and by various agents that activate the cAMP-dependent protein kinase pathway (Smas and Sul, 1995 supra; Morrison and Framer, 2000 supra; Ntambi and Kim, 2000 supra). Inhibition of these pathways and/or their downstream effectors such as transcription factors PPARγ and C/EBPα thus provide potential targets for modulating adipocyte differentiation and maturation. Conversely, various growth factors and cytokines including tumour necrosis factor α (TNFα), epidermal growth factor (EGF), basic fibroblast growth factor (bFGF) and transforming growth factor β (TGFβ) as well as activators of protein kinase C and calcium ionophores can lead to rapid down regulation of PPARγ and inhibition of mature adipocyte differentiation (Smas and Sul, 1995 supra; Xing et al, Endocrinology 138(7):2776-2783, 1997). Down regulation of the EGF repeat domain-containing transmembrane protein preadipocyte factor-1 (pref-1) in mature adipocytes has been shown to be associated with preadipocyte differentiation and drugs such as statins which induce high pref-1 levels have been shown to inhibit adipocyte differentiation (Nicholson et al, Br. J. Pharmacol. 151:807-815, 2007). Collectively, the cytokine and growth factor receptors and binding sites involved in inhibiting adipocyte differentiation and maturation also provide potential targets for therapeutic modulation of adipocyte function.

There is an urgent need to control obesity. Current weight control measures that include multiple drug therapies, surgery, regular exercise and diet can be invasive, time consuming, expensive and not always appropriate for some individuals.

The proteins, α-casein, β-casein and κ-casein are known to be one of the most nutritive groups of proteins in milk, containing all of the common amino acids and rich in essential ones.

α-Casein [(S1) 23 kDa and (s2) 25 kDa], β-casein (24 kDa) and κ-casein (19 kDa) are the most abundant proteins found in milk accounting for 80% of total protein. There have been a number of reports of the bioactive components from the casein proteins. Predominantly, the bioactive peptides from α-casein and β-casein peptide fragments have recorded anti-proliferative, pro-apoptotic, ACE-inhibitor, immunomodulatory, opioid antagonistic and pro-proliferative properties, whereas κ-casein bioactive peptides have been shown to be anti-thrombotic and opioid antagonistic (Meisel, Curr Med. Chem. 12(16):1905-1919, 2005; Meisel and Bockelmann, Antonie van Leeuwenhoek 76(1-4):207-215, 1999). Caseins are a slow-digesting source of amino acids as opposed to the fast-digesting whey protein, and provide an extremely high source of glutamine (post-workout muscle building supplements). However, neither α-casein nor κ-casein or a purified milk fraction containing these proteins has been shown to inhibit adipocyte differentiation or contribute to control of obesity.

Zemel et al (US Patent Application No. 2004/0197382) have proposed inducing the loss of adipose tissue by providing a high calcium diet. In one aspect, the high calcium is provided in the form of dairy products. Ward et al (US Patent Application No. 2003/0165574) suggest that a mixture of milk minerals plus a protein source (a specific casein fragment) may cause weight loss. The casein fragment is proposed to act on gastrointestinal release of CCK to modulate satiety.

Lactoperoxidase is a member of the whey protein family, the second most abundant group of milk proteins. Whey constitutes 20% of total protein in milk. Lactoperoxidase is a 78 kDa heme containing oxidation-reduction enzyme present in milk and secretory glands. Lactoperoxidase consists of a single polypeptide chain of 612 amino acid residues, four or five carbohydrate chains constituting a total of approximately 10% of the molecular weight and accounts for 0.25-0.5% of total protein found in whey. Lactoperoxidase is a highly useful protein, as it has anti-microbial activity which permits its use as a fungicide, viricide, protozoacide and bacteriocide both in products which need preservatives and in therapeutic products (Meisel, 2005 supra; Marshall, Altern Med. Rev. 9(2):136-156, 2004). In addition to these anti-microbial effects, bovine lactoperoxidase is known to catalyze the oxidation of a number of organic molecules such as thiols, phenols, catecholamines, steroid hormones, halides and nitrite (Tanaka et al, Biosci. Biotecnol. Biochem. 67(10):2254-2261, 2003). However, neither lactoperoxidase nor a fragment or fragments of lactoperoxidase or purified milk fractions containing lactoperoxidase has been shown to inhibit adipocyte differentiation or contribute to control of obesity.

Whey is a collection of globular proteins that result as a by-product from cheese production. Whey is known to have bioactive compounds and has known nutritional benefits. In milk, there are a number of glycoproteins which include lactoferrin, secretory IgA, IgG, IgM, free secretory component (FSC), and milk mucin. Glycoproteins isolated from mammals have lipid mobilizing properties as outlined by Tisdale and Todorov (WO/1999/062939) characterizing Zn-α-glycoprotein as a therapeutic derived from a murine tumor to possibly control obesity. In addition, Kodama and Kimura (U.S. Pat. No. 6,828,298) have specifically isolated a glycoprotein from milk whey and albumen from chicken eggs which inhibits Helicobacter pylori colonization. A method for preparing mucin enriched glycoproteins from milk has previously been described that comprises a complex multi-step process involving heating steps that may lead to unwanted protein denaturation and loss of some biological activity. See International Patent Application No. PCT/US2002/010485.

It is proposed herein to use milk products and whey-derived products in a nutraceutical and/or therapeutic approach to controlling obesity by inhibiting adipogenesis and its related processes.

### SUMMARY

The present invention is predicated in part on the identification of regulators of adipogenesis from milk fractions including whey. The regulators are, in particular, inhibitors of adipogenesis. The identification of the adipogenic inhibitors enables a nutraceutical and/or therapeutic approach to controlling obesity and complications arising therefrom including diabetes, hypertension and heart disease. The inhibitors identified from milk or whey comprise one or more glycoproteins from or a fraction comprising glycoproteins or crypteins or muteins of any of these glycoproteins. Purified milk fractions are also provided which comprise inhibitors of adipogenesis, the fractions being those which have α-casein, κ-casein or lactoperoxidase. Mimetics of these regulators of adipogenesis are also contemplated herein. In particular, a method for production of a specific glycoprotein fraction isolated from whey is provided that has the ability to inhibit adipocyte differentiation and lipid accumulation as well as inhibiting or reducing weight gain and detrimental changes in metabolic parameters associated with a high fat diet. It is proposed to utilize the adipogenic inhibitors in nutraceutical and pharmaceutical formulations, as food additives and supplements and as targets for the generation of mimetics. A method for isolating the bioactive glycoprotein fraction from whey and other sources is also provided, the glycoproteins having adipogenic controlling properties

Accordingly, one aspect herein contemplates a method for inhibiting adipogenesis or a process associated with adipogenesis in a subject, the method comprising administering to the subject an effective amount of an adipogenesis regulator, the regulator of adipogenesis being a glycoprotein from whey or a whey fraction comprising one or more glycoproteins or a cryptein or mutein of one or more of these proteins or is a milk-derived fraction comprising α-casein, κ-casein and/or lactoperoxidase.

Another aspect of the present invention provides a method for treating or preventing obesity or a complication arising therefrom in a subject, the method comprising administering to the subject an effective amount of an adipogenesis regulator, the regulator of adipogenesis being a glycoprotein from whey or a whey fraction comprising one or more glycoproteins or a cryptein or mutein of one or more of these glycoproteins or a milk-derived fraction comprising α-casein, κ-casein and/or lactoperoxidase.

Still another aspect of the present invention is directed to the use of a regulator of adipogenesis being a glycoprotein from whey or a whey fraction comprising one or more glycoproteins or a cryptein or mutein of one or more of these glycoproteins or is a milk-derived fraction containing α-casein, κ-casein and/or lactoperoxidase in the manufacture of a nutraceutical, or food supplement to control obesity in a subject.

Even yet another aspect of the present invention provides for the use of a regulator of adipogenesis being a glycoprotein from whey or a whey fraction comprising one or more glycoproteins or a cryptein or mutein of one or more of these glycoproteins or is a milk-derived fraction comprising α-casein, κ-casein and/or lactoperoxidase in the manufacture of a medicament to control obesity in a subject.

Another aspect of the present invention contemplates a nutraceutical formulation comprising a regulator of adipogenesis being a glycoprotein from whey or a whey fraction comprising one or more glycoproteins or a cryptein or mutein of one or more of these glycoproteins or a milk-derived fraction comprising α-casein, κ-casein and/or lactoperoxidase.

A therapeutic composition when used in the treatment of obesity or a complication arising therefrom in a subject, the composition comprising a regulator of adipogenesis being a glycoprotein from whey or a whey fraction comprising one or more glycoproteins or a cryptein or mutein of one or more of these glycoproteins or a milk-derived fraction comprising α-casein, κ-casein and/or lactoperoxidase, the composition further comprising one or more pharmaceutically acceptable carriers, diluents and/or excipients.

Even yet another aspect of the present invention is directed to a food additive or supplement which reduces adipogenesis in a subject, the additive or supplement comprising a regulator of adipogenesis being a glycoprotein from whey or a whey fraction comprising one or more glycoproteins or a cryptein or mutein of one or more of these glycoproteins or a milk-derived fraction comprising α-casein, κ-casein and/or lactoperoxidase.

A further aspect of the present invention relates to a method for enriching whey for glycoproteins and proteins, the method comprising subjecting the whey to a single multi-lectin fractionation, the lectins selected from concanavalin A, jacalin and wheat germ agglutinin or at least one other lectin substituting for concanavalin A, jacalin or wheat germ agglutinin and then screening a fraction or isolated molecule resulting therefrom for a desired activity such as adipogenesis regulating activity.

The formulations may be administered in a number of convenient ways such as by oral, subcutaneous, intravenous or rectal administration.

### BRIEF DESCRIPTION OF THE FIGURES

FIG. 1 is a photographic representation of SDS-PAGE analysis of bovine milk-derived whey and MLAC whey glycoprotein fraction. Lane 1) Molecular weight markers; Lane 2) 20 μg whey; Lane 3) 20 μg MLAC whey glycoprotein fraction eluted with acetic acid. (Abbreviations MLAC whey, glycoprotein fraction from whey).

FIG. 2 is a photographic representation showing adipogenesis assay data following treatment of cells with dairy-derived protein preparations A0 to H0 and cryptein library D5. A0 to H0 represent chromatographically purified preparations of major bovine milk-derived proteins ranging in purity from approximately 70-90%). All libraries are assayed at a final concentration of 0.5 mg/ml. (Abbreviations: A, α-lactalbumin; B, β-lactoglobulin; C, lactoferrin; D, lactoperoxidase; E, α-casein; F, β-casein; G, κ-casein and H, whey protein; 0, native protein; 5, 2 hr peptic digest; MDI; combination of methylisobutylxanthine, dexamethsaone and insulin).

FIG. 3 is a graphical representation of adipogenesis assay data following treatment of cells with various concentrations of a purified MLAC whey fraction. Adipocyte differentiation and lipid accumulation was quantitatively measured using Nile Red fluorescence. (Abbreviations: MDI, combination of methylisobutylxanthine dexamethasome and insulin; MLW, glycoprotein fraction from whey; TNF, tumour necrosis factor). The MLW fraction is a potent inhibitor of adipogenesis over a broad concentration range.

FIG. 4 is a photographic representation of a Western blot analysis of the key adipocyte transcription factor PPARγ. Preadipocyte cultures were differentiated in the presence of an MLAC whey preparation at a final concentration of 300, 100 and 33 μg/ml. (Abbreviations: Con, control; MDI, combination of methylisobutylxanthine dexamethasome and insulin; MLAC whey, glycoprotein fraction from whey; PPARγ; peroxisome proliferator-activated receptor γ; TNF-α, tumour necrosis factor-α).

FIG. 5 is a graphical representation of the effects of low and high dose of LAP001 administration in a high fat diet animal model. Rats fed a high fat diet over 28 days were treated by constant subcutaneous infusion via mini-pumps with either Vehicle, low dose LAP001 (0.5 mg/rat/day) or high dose LAP001 (2.5 mg/rat/day). A) mean weekly body weight (grams) over 4 weeks; B) mean change in body weight (grams) (change from baseline to day 28); C) mean change in blood glucose levels (mmol/L) (from baseline to day 28); D) mean plasma insulin levels (ng/mL); E) mean plasma adiponectin levels (ng/mL) and F) mean plasma leptin levels (ng/mL) in low dose (0.5 mg/rat/day) and high dose (5 mg/rat/day) LAP001 treated Sprague Dawley rats fed a high fat diet compared to vehicle (n=10 rats per group). All variables are expressed as mean±standard error of the mean (SEM) or mean difference±standard error of the difference (SED). Statistical analysis of the data was analysed by a multifactor repeated measure Analysis of Variance (ANOVA). *: P<0.05; ***: P<0.001.

### DETAILED DESCRIPTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Reference to any prior art in this specification is not, and should not be taken as, an acknowledgment or any form of suggestion that this prior art forms part of the common general knowledge in any country.

As used in the subject specification, the singular forms “a”, “an” and “the” include plural aspects unless the context clearly dictates otherwise. Thus, for example, reference to “a glycoprotein” includes a single glycoprotein, as well as two or more glycoproteins; reference to “an adipogenesis inhibitor” includes a single adipogenesis inhibitor, as well as two or more adipogenesis inhibitors; reference to “the invention” includes a single or multiple aspects of an invention, and so forth.

Embodiments disclosed and described herein relate to the identification of proteins, including glycoproteins and crypteins and muteins thereof and fractions comprising one or more proteins, including glycoproteins and/or crypteins or muteins thereof from milk or milk product which inhibit adipogenesis in a subject. Alternative sources of the proteins are also contemplated herein (e.g. soy milk). Particular fractions are whey-derived glycoprotein fractions generated by a single multi-lectin fractionation and which exhibit the antiadipogenic activity. Other fractions include milk fractions which can be defined as a fraction comprising α-casein, κ-casein and/or lactoperoxidase, even if these proteins are not themselves the bioactive glycoprotein.

Reference to “inhibit” in relation to adipogenesis includes retarding or otherwise delaying or reducing adipogenesis as well as processes associated with adipogenesis. The inhibition may be transient or permanent and may be total inhibition or partial inhibition. Adipogenesis includes the differentiation of pre-adipocytes into mature adipocytes. Hence, reference to inhibition of adipogenesis can be considered, in one embodiment, as the inhibition of the differentiation process. This can be measured ex vivo from a subject or in an in vitro assay. Notwithstanding, embodiments herein encompass the inhibition of lipogenesis which is a metabolic process of fat deposit formation. Hence, inhibition of a process associated with adipogenesis includes lipogenesis.

Accordingly, one aspect herein contemplates a method for inhibiting adipogenesis or a process associated with adipogenesis in a subject, the method comprising administering to the subject an effective amount of an adipogenesis regulator, the regulator of adipogenesis being a glycoprotein from whey or a whey fraction comprising one or more glycoproteins or a cryptein or mutein of one of these glycoproteins or a milk-derived fraction comprising α-casein, κ-casein and/or lactoperoxidase.

Milk or a milk by-product or whey is subjected to fractionation and/or separation means to generate groups of proteins or single proteins that have an inhibitory effect on adipogenesis in an in vitro screening assay. The fractionation and separation processes includes, but not limited to, ion exchange chromatography, size-based chromatography, affinity-binding chromatography including lectin-binding and immunoaffinity chromatography as well as protease-based or chemical-based fractionation and separation to generate peptides referred to herein as “crypteins”. The generation of crypteins is disclosed in International Publication No. 2004/008148 and reviewed by Autelitano et al, Drug Discov. Today 11(7/8):306-314, 2006 which is incorporated herein by reference.

Reference to a “whey fraction” includes the fraction of whey which comprises one or more glycoproteins which are bioactive in an adipogenesis inhibition assay. The present invention from a milk fraction or product which comprises one or more of α-casein and/or κ-casein and/or lactoperoxidase such milk fractions or products are proposed to contain the bioactive molecule of the present invention. However, the bioactive molecule is not necessarily one of α-casein, κ-casein or lactoperoxidase.

The proteins or peptides or fractions or crypteins obtained may also be subjected to random or site-directed mutagenesis to generate mutated molecules referred to herein as “muteins”. A “protein” herein includes a “glycoprotein”. The protein may also be more commonly regarded as a peptide (or glycopeptide).

Hence, in an embodiment, bioactive molecules are identified on the basis of an in vitro assay of adipogenesis and are referred to herein as “adipogenesis regulators”, “adipogenesis modulators”, “adipogenesis inhibitors”, “adipogenesis medicaments” or other like terms. Notwithstanding the adipogenesis regulators may also have other biological activities such as inhibiting lipogenesis.

A method is provided for identifying an adipogenesis regulator from whey or milk or a milk product, the method comprising providing a library of proteins, protein-containing fractions, crypteins and/or muteins from the whey or milk or milk product and screening the library for an ability to inhibit adipogenesis in an in vitro assay, optionally subjecting protein or cryptein or mutein fractions to separation means to identify particular molecules or groups of molecules or sub-fractions which inhibit adipogenesis and then identifying the adipogenesis regulator as an isolated protein, sub-fraction, cryptein or mutein.

As indicated above, the library is generated by fractionation of whey or milk or a milk products by any one of a number of chromatographic and molecule-separating methods. Particular methods include ion exchange chromatography, affinity chromatography such as immunoaffinity chromatography as well as lectin-based affinity chromatography. In one particular embodiment the whey is subjected to lectin-based affinity chromatography comprising three lectins, concanavalin A, jacalin and wheat germ agglutinin (Yang and Hancock, J Chromatography A 1053: 79-88, 2004) and whey-derived glycoproteins are concentrated using a single step elution with 0.1 M acetic acid.

Any one or more of the above-listed lectins may be replaced by another lectin. Other lectins which may be employed include peanut lectin (PNA), lentil lectin (LCA), Lens culinaris agglutinin (LCA), Griffonia (Bandeiraea) simplicifolia lectin II (GSLII), Aleuria aurantia lectin (AAL), Hippeastrum hybrid lectin (HHL,AL), Sambucus nigra lectin (SNA,EBL), Maackia amurensis lectin II (MAL II), Ulex europaeus agglutinin I (UEA I), Lotus tetragonolobus lectin (LTL), Galanthus nivalis lectin (GNL), Euonymus europaeus lectin (EEL) and Ricinus communis agglutinin I (RCA).

The assay to identify the adipogenesis regulators, in a particular embodiment, comprises subjecting a protein source such as but not limited to whey or milk or milk product to digestion, cleavage and/or reduction to generate a series of digested or reduced fractions, subjecting each fraction to an adipogenesis assay to identify fractions having adipogenesis regulating activity, and then optionally subjecting the active fractions to separation or purification means to generate single proteins, crypteins, muteins or enriched sub-fractions having adipogenesis regulator activity. As indicated above, an adipogenesis regulator includes, in one embodiment, a regulator of lipogenesis.

The term “protein” as used herein shall be taken to refer to any polymer consisting of amino acids linked by covalent bonds and this term includes within its scope parts or fragments of full length proteins, such as, for example, polypeptides, peptides and shorter peptide sequences consisting of at least two amino acids, more particularly at least about 5 amino acid residues. The term “protein” includes all moieties containing one or more amino acids linked by a peptide bond. In addition, this term includes within its ambit polymers of modified amino acids, including amino acids which have been post-translationally modified, for example by chemical modification including but not restricted to glycosylation, phosphorylation, acetylation and/or sulphation reactions that effectively alter the basic peptide backbone. Accordingly, a protein herein may be derived from a naturally-occurring protein, and in particular may be derived from a full-length protein by chemical or enzymatic cleavage, using reagents such as CNBr, or proteases such as trypsin or chymotrypsin, amongst others. Alternatively, such peptides may be derived by chemical synthesis using well known peptide synthetic methods. In another alternative, the proteins are isolated following, for example, ion exchange or affinity chromatography.

Also included within the scope of the definition of a “protein” are amino acid sequence variants (referred to herein as “muteins”). These may contain one or more amino acid substitutions, deletions, or insertions in a naturally-occurring amino acid sequence. Such muteins may be synthesized by chemical peptide synthesis. Amino acid substitution reactions are well-known in the art. Rules for making such substitutions are well known. More specifically, conservative amino acid substitutions are those that generally take place within a family of amino acids that are related in their side chains. Genetically-encoded amino acids are generally divided into four groups: (1) acidic=aspartate, glutamate; (2) basic=lysine, arginine, and histidine; (3) non-polar=alanine, valine, leucine, isoleucine, proline, phenylalanine, methionine, and tryptophan; and (4) uncharged polar=glycine, asparagine, glutamine, cysteine, serine, threonine, and tyrosine. Phenylalanine, tyrosine and tryptophan are also jointly classified as aromatic amino acids.

Included within the scope of the definition of a “protein” are muteins which have undergone unnatural modifications such as but not limited to protection, carboxylation, and derivatization by amide and non-amide bonds as well as covalent and non-covalent modification.

The term “protein” also includes recombinantly generated polypeptides, oligopeptides or shorter peptide sequences. Protein which is produced in vitro or in a host cell by the expression of a genetic sequence encoding the protein, which genetic sequence is under the control of a suitable promoter, wherein a genetic manipulation has been performed in order to achieve said expression. Accordingly, the term “recombinant protein” clearly encompasses proteins produced by the expression of genetic sequences contained in viral vectors, cosmids or plasmids that have been introduced into prokaryotic or eukaryotic cells, tissues or organs. Genetic manipulations which may be used in this context will be known to those skilled in the art and include, but are not limited to, nucleic acid isolation, restriction endonuclease digestion, exonuclease digestion, end-filling using the Klenow fragment of E. coli DNA polymerase I to T4 DNA polymerase enzymes, blunt-ending of DNA molecules using T4 DNA polymerase or ExoIII enzymes, site-directed mutagenesis, ligation, and amplification reaction.

In the method herein, the initial library of proteins comprises a heterogeneous and unfractionated mixture of proteins derived from a precursor protein source (or protein mixture or protein-containing biological extract) such as whey or milk which provides a comprehensive range of potentially bioactive proteins or crypteins or muteins.

The library is conveniently subjected to initial analysis or characterization to provide information on the activity or size or other characteristics of the component proteins, for example by adipogenesis assay or matrix assisted laser desorption time of flight mass spectrometry (MALDI-ToF MS).

Initial screening of the library to confirm that it includes bioactive proteins may particularly be carried out using an in vitro adipogenesis assay.

After the library has been confirmed as including bioactive proteins, it is fractionated by suitable means of fractionation including but not limited to chromatographic methods such as, but not limited to, ion exchange, size exclusion, hydrophobic interaction and/or reverse phase-high performance liquid chromatography, field-flow fractionation (including but not limited to sedimentation, flow, thermal and steric), and electrophoresis in order to provide fractions of the library for subsequent further screening. This further screening may be carried out by any suitable screening assay or assays as discussed above so as to identify an active fraction or active fractions which include bioactive proteins.

Since such active fractions may include more than one protein, each fraction may, if desired, be subjected to one or more further cycles of fractionation by suitable means of fractionation including but not limited to chromatography, field-flow fractionation (including but not limited to sedimentation, flow, thermal and steric), and electrophoresis to form sub-fractions, followed by screening of each sub-fraction as described above so as to identify an active sub-fraction or active sub-fractions which include bioactive proteins (i.e. sub-fractions or proteins which have adipogenesis regulator activity).

Each fraction or sub-fraction which is produced may also be subjected to analysis or characterization as described above, for example by adipogenesis assay or MALDI-ToF MS, so as to provide information on the activity, size or other characteristics of the component proteins in the fraction or sub-fraction.

Using this approach, milk-derived fractions containing α-casein, κ-casein and lactoperoxidase as well as crypteins and muteins thereof and fractions comprising same and whey glycoproteins and fractions comprising same as well as crypteins and muteins of the whey glycoproteins are proposed to be adipogenesis regulators useful in the inhibition of adipogenesis. As indicated above, regulators of adipogenesis include, in an embodiment, regulators of lipogenesis or other metabolic processes associated with obesity.

The source of the milk is not critical to the practice of the embodiments herein and may be from any mammal including a human, cow, sheep, goat, horse, pig, camel, laboratory test animal (e.g. mouse, rat, rabbit, guinea pig), companion animal (e.g. dog, cat) or captive wild animal (e.g. elephant, zebra, kangaroo). Once the protein or fraction has been identified as an inhibitor of adipogenesis it may also be sourced elsewhere from milk. The term “milk” includes soy milk and other legume milks.

The identification of proteins which inhibit adipogenesis enables their use to treat or prevent obesity and complications arising therefrom such as diabetes, hypertension and heart disease.

Accordingly, another aspect of the present invention provides a method for treating or preventing obesity or a complication arising therefrom in a subject, the method comprising administering to the subject an effective amount of an adipogenesis regulator, the regulator of adipogenesis being a glycoprotein from whey or a whey fraction comprising one or more glycoproteins or a cryptein or mutein of one or more of these glycoproteins or a milk-derived fraction comprising α-casein, κ-casein and/or lactoperoxidase.

In a related embodiment, provides for the use of a regulator of adipogenesis being a glycoprotein from whey or a whey fraction comprising one or more glycoproteins or a cryptein or mutein of one or more of these glycoproteins or a milk-derived fraction comprising α-casein, κ-casein and/or lactoperoxidase.

The “subject” as used herein refers to an animal, particularly a mammal and more particularly a primate including a lower primate and even more particularly a human who can benefit from the formulations and methods of the present invention. A subject regardless of whether a human or non-human animal may be referred to as a subject, an individual, patient, animal, host or recipient. The formulations and methods of the present invention have applications in human medicine, veterinary medicine as well as in general, domestic or wild animal husbandry. Hence, the formulations described herein are applicable for the treatment of humans, livestock animals, racing industry animals and companion animals.

As indicated above, particular subjects are humans, non-human primates such as marmosets, baboons, orangutangs, lower primates such as tupia, livestock animals, laboratory test animals, companion animals or captive wild animals. A companion animal includes a dog and cat. A human is the most preferred target. However, non-human animal models may be used.

Examples of laboratory test animals include mice, rats, rabbits, guinea pigs and hamsters. Rabbits and rodent animals, such as rats and mice, provide a convenient test system or animal model as do primates and lower primates. Livestock animals include sheep, cows, pigs, goats, horses and donkeys. Racing industry animals include horses, camels and dogs.

The adipogenic regulators herein may be formulated from any convenient manner such as in a nutraceutical formulation or a pharmaceutical formulation using standard formulation technology.

Another aspect of the present invention contemplates a nutraceutical formulation comprising a regulator of adipogenesis being a glycoprotein from whey or a whey fraction comprising one or more glycoproteins or a cryptein or mutein of one or more of these glycoproteins or a milk-derived fraction comprising α-casein, κ-casein and/or lactoperoxidase.

A therapeutic composition when used in the treatment of obesity or a complication arising therefrom in a subject is provided herein, the composition comprising a regulator of adipogenesis being a glycoprotein from whey or a whey fraction comprising one or more glycoproteins or a cryptein or mutein of one or more of these glycoproteins or a milk-derived fraction comprising α-casein, κ-casein and/or lactoperoxidase, the composition further comprising one or more pharmaceutically acceptable carriers, diluents and/or excipients.

Even yet another aspect of the present invention is directed to a food additive or supplement which reduces adipogenesis in a subject, the additive or supplement comprising a regulator of adipogenesis being a glycoprotein from whey or a whey fraction comprising one or more glycoproteins or a cryptein or mutein of one or more of these glycoproteins or a milk-derived fraction comprising α-casein, κ-casein and/or lactoperoxidase.

Embodiments herein further contemplate a method of isolating a glycoprotein or fraction of glycoproteins from milk, the method comprising:

(a) contacting a composition, comprising at least, but not limited to, three different lectins attached to a solid support, with a sample containing the glycoproteins, under conditions that promote binding of glycoproteins in the sample to the lectins, thereby providing a bound sample;

(b) removing unbound sample from the contacted composition; and

(c) eluting at least one glycoprotein from the bound sample,

wherein the glycoprotein is in the eluted sample.

In one embodiment, the lectins are concanavalin A, jacalin and wheat germ agglutinin.

Other lectins which may be employed include peanut lectin (PNA), lentil lectin (LCA), Lens culinaris agglutinin (LCA), Griffonia (Bandeiraea) simplicifolia lectin II (GSLII), Aleuria aurantia lectin (AAL), Hippeastrum hybrid lectin (HHL,AL), Sambucus nigra lectin (SNA,EBL), Maackia amurensis lectin II (MAL II), Ulex europaeus agglutinin I (UEA I), Lotus tetragonolobus lectin (LTL), Galanthus nivalis lectin (GNL), Euonymus europaeus lectin (EEL), and Ricinus communis agglutinin I (RCA). Any three of the above other lectins may be employed or at least one of concanavalin A, jacalin and wheat germ agglutinin may be substituted by at least one of the other lectins.

Kits are contemplated herein such as comprising compartments or containers each containing a glycoprotein from whey or a whey fraction comprising one or more glycoproteins or a cryptein or mutein of one or more of these glycoproteins or a milk-derived fraction comprising α-casein, κ-casein and/or lactoperoxidase. The kits may be packaged for sale as food additives or supplements or may be in the form of nutraceutical or pharmaceutical packs. For example, the proteins may be in a freeze dried form, the contents of which may be reconstituted prior to use in food or as a medicament.

The present invention further provides a method for inhibiting adipogenesis or a process associated with adipogenesis in a subject, the method comprising administering to the subject an effective amount of an adipogenesis regulator, the regulator of adipogenesis selected from the list consisting of a glycoprotein from whey and a whey fraction comprising one or more glycoproteins or a cryptein or mutein of one or more of these glycoproteins.

Yet another aspect of the present invention provides a method for inhibiting adipogenesis or a process associated with adipogenesis in a subject, the method comprising administering to the subject an effective amount of an adipogenesis regulator, the regulator consisting of a milk-derived fraction comprising α-casein, κ-casein and/or lactoperoxidase.

The formulations described herein are also contemplated when used for the treatment of obesity or complications arising therefrom such as hypertension, diabetes or heart disease.

Administration of the formulations of the present invention may be any convenient route such as by oral, subcutaneous and subcutaneous mini-pump, intravenous or rectal administration. Formulations suitable for oral, subcutaneous, intravenous or rectal administration are referred to as oral, subcutaneous, intravenous or rectal formulation.

The present invention is further described by the following non-limiting Examples.

### Example 1

### Whey Glycoprotein Purification

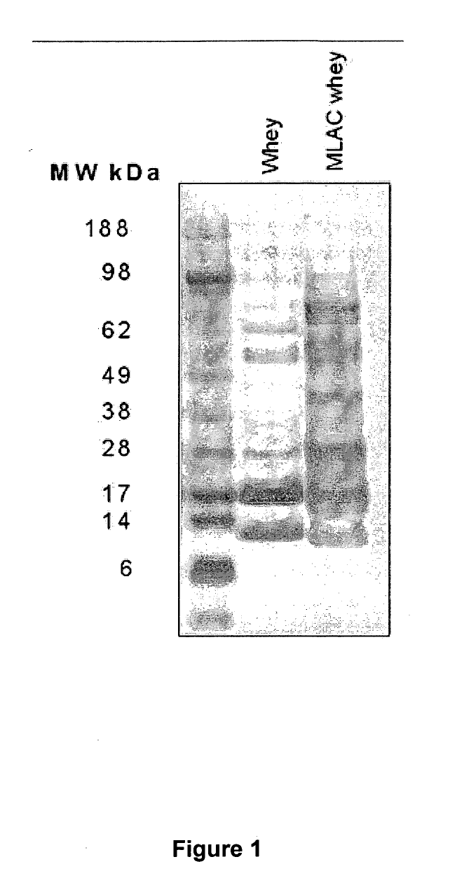

FIG. 1 shows SDS-PAGE analysis of bovine milk whey and whey glycoproteins (MLAC whey) samples against molecular weight samples. SDS-PAGE was performed using 4-12% w/v Bis-Tris gels on a Novex NuPAGE system. The proteins are resolved with the NuPAGE MOPS SDS running buffer in a Novex Mini-Cell system at 200 volts. The proteins are visualized by staining with Simply Blue safe stain blue. MLAC whey is a fraction of whey glycoprotein obtained following multi-lectin chromatography enrichment using lectins concanavalin A, jacalin and wheat germ agglutinin. The bound glycoproteins were eluted with acetic acid (Lane 3) to provide an enriched whey glycoprotein fraction. The eluted glycoproteins range in molecular weight from less than 14 kDa to greater than 100 kDa. There is clear visible enrichment of glycoproteins (Lane 3) using multi-lectin chromatography when compared to whey starting material (Lane 2).

Whey samples were loaded on a multi-lectin affinity column packed with equal amounts of lectins concanavalin A, jacalin and wheat germ agglutinin. After a 15 minute reaction, the unbound proteins are eluted with 10 ml of equilibration buffer and the captured proteins are released with 12 ml of elution buffer comprised of 0.1 M acetic acid. The flow-through and eluted fractions were both collected and concentrated with 5 kDa Amicon filters. The total amount of protein loaded on the column, the amount of protein collected in the flow-through and the amount of protein collected in eluted fraction were measured using a BCA protein assay. The flow-through and eluted fractions are analysed by mass spectrometry and the eluted proteins are assayed.

### Example 2

### Screening of Milk-Derived Protein Fractions in an Adipogenesis Assay

Various chromatographically purified bovine milk-derived proteins were subjected to peptic digestion as described in WO2004/008148 and both undigested and digested fractions were assayed in an adipogenesis assay.

The assay is described in Student et al, 1980 supra that has been modified and described below and in Example 3. All fractions (referred to herein as “libraries”) are assayed at a final concentration of 0.5 mg/ml.

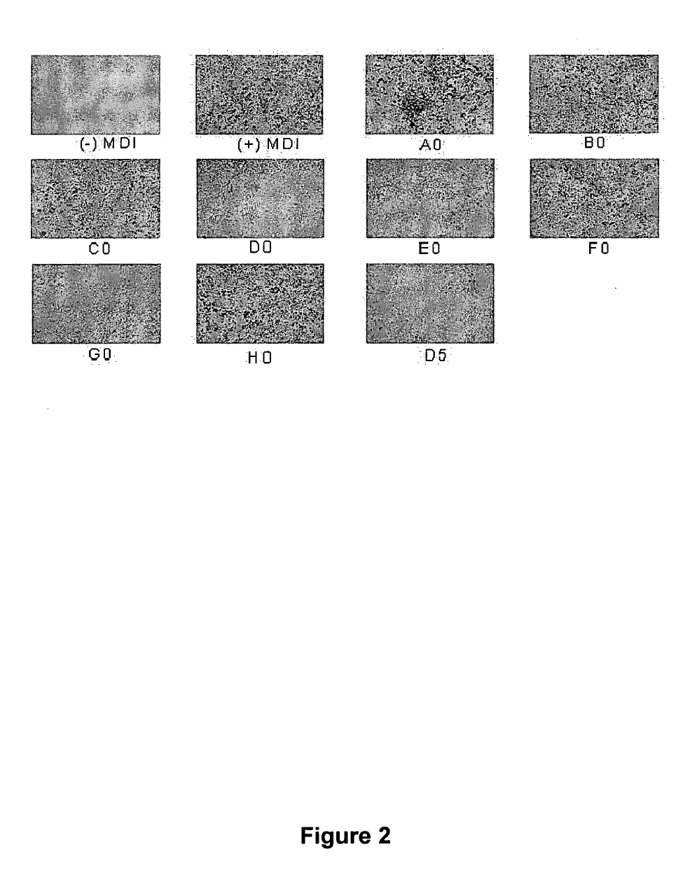

The results (shown in FIG. 2) demonstrated that components of the purified protein preparations comprising α-casein (E0), κ-casein (G0), lactoperoxidase (D0) and lactoperoxidase cryptein library (D5) acted as inhibitors of adipogenesis. The presence of anti-adipogenesis bioactivity in multiple milk-derived chromatographic fractions indicates that low concentrations of the bioactive components co-purify in fractions containing several major milk proteins. Mouse preadipocytes (3T3-L1) were used to determine the effects of dairy protein-derived compounds on the differentiation to mature adipocytes and accumulation of intracellular lipid stained with Oil Red O. Preadipocytes were routinely passaged in Growth Medium (DMEM, 10% v/v bovine calf serum, penicillin, streptomycin, amphotericin B) and were plated in a volume of 100 μl in this medium in 96 well microplates at a density of 10,000 cells per well for assay. Preadipocytes were grown at 37° C. for 3 days in a humidified atmosphere containing 5% CO₂ to reach a post-confluent state. Medium was then removed and replaced with 100 μl of either fresh Growth Medium for control preadipocyte wells or with Induction Medium (DMEM, 10% fetal bovine serum, 0.5 mM IBMX (methyl-3-isobutylxanthine), 10 μg/ml insulin, 1 μM dexamethasone, penicillin, streptomycin, amphotericin B) to induce differentiation to mature adipocytes. As a control for inhibition of adipocyte differentiation, four wells in each assay plate were treated with 5 ng/ml TNF-α which was added concurrently with Induction Medium. Dairy protein-derived compounds were also added concurrently with Induction Medium to test their effects on modulating differentiation of preadipocytes to mature, lipid-laden adipocytes. Assay plates were routinely allowed to differentiate for 6-7 days, during which time wells were monitored by phase contrast microscopy to document the altered cellular phenotype and accumulation of refractive lipid vesicles. Medium was then removed from the assay plates and cells were fixed in 1% v/v paraformaldehyde in phosphate buffered saline for 20 minutes at room temperature. The fixative was aspirated, wells were washed twice with 200 ml PBS and prepared for Oil Red 0 staining using reagents from an Adipogenesis Kit (Chemicon). Each well was stained with 200 ml PBS containing 50 μl Oil Red O solution for 15 minutes at room temperature. Stain was removed and wells were washed three times with 100 μl Wash solution before being examined by light microscopy to visualize the extent of cellular differentiation and accumulation of lipid vesicles. The figure shows typical responses when preadipocytes undergo induction in the presence of various dairy protein-derived fractions (A0-H0 and D5). Preadipocytes induced in the presence of fractions D0, E0, G0 and D5 fail to fully differentiate and display substantially reduced levels of Oil Red O stained intracellular lipids. (−) MDI and (+) MDI refer to control wells treated without or with adipocyte Induction Medium containing Methyl-3-isobutylxanthine, Dexamethasone and Insulin.

### Example 3

### MLAC Whey Glycoprotein Fraction Potently Inhibits Adipogenesis

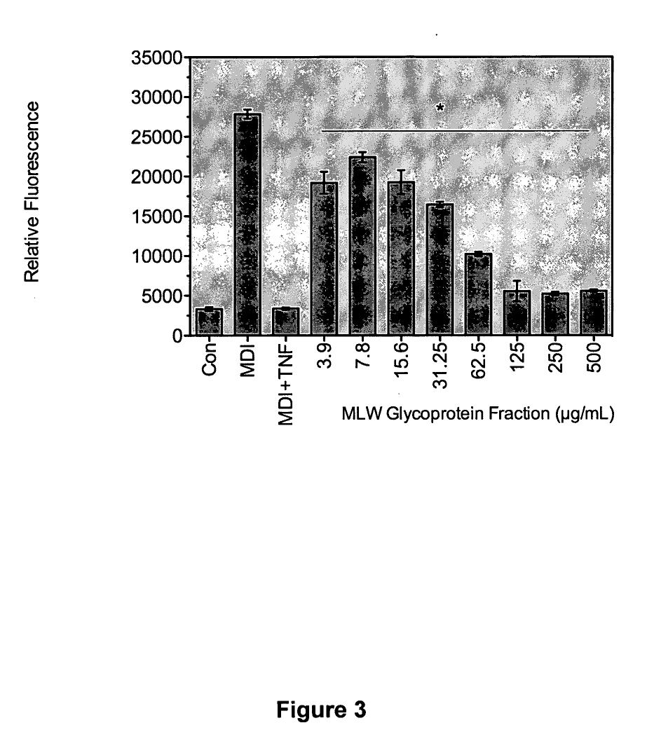

MLAC whey glycoprotein fraction prepared as described in Example 1 was tested for its ability to modulate adipogenesis in an in vitro assay and it was found that this fraction highly enriches the anti-adipogenesis bioactivity (FIG. 3). To quantify the effect of this dairy-derived fraction on the differentiation to mature adipocytes and accumulation of intracellular lipid, an identical strategy was employed to grow and treat 3T3-L1 preadipocytes as outlined in Example 2. Instead of staining lipid droplets with Oil Red O, intracellular lipid droplets were stained with the vital stain Nile Red and lipid accumulation was quantitated by measuring the subsequent fluorescence. Cultures were treated with a range of concentrations of MLAC whey derived fraction from 3.9-500 μg/ml. Following differentiation for 6-7 days as described, media was removed from wells, cells were washed with 200 μl PBS and replaced with 200 μl fresh PBS. The plate was then transferred to a Polarstar multi-mode plate reader (BMG) equipped with automatic injectors. Each well was injected with 5 μl Nile Red dye solution (AdipoRed reagent from Cambrex) followed by a 2 second shake. The plate was then incubated at room temperature for 10 minutes before reading fluorescence at Ex 485 nm/Em 590 nm. The data shown represents relative fluorescence output as a measure of intracellular lipid accumulation as determined by Nile Red fluorescence assay. Con and MDI refers to control wells treated without or with adipocyte Induction Medium containing Methyl-3-isobutylxanthine, Dexamethasone and Insulin. TNF refers to 5 ng/ml TNF-β used as a control inhibitor of adipocyte differentiation. The MLAC whey fractions (MLW) inhibited adipogenesis in a dose-dependent manner that was significantly different to the MDI group across a broad concentration range (FIG. 3). Bars represent mean±SEM (n=4 for control wells and n=3 for each MLW concentration tested). *: P<0.05 vs MDI group (one way ANOVA followed by Newman-Keuls comparison).

### Example 4

### Dairy-Derived Purified MLAC Whey Fraction Inhibits Signalling Associated with Adipogenesis

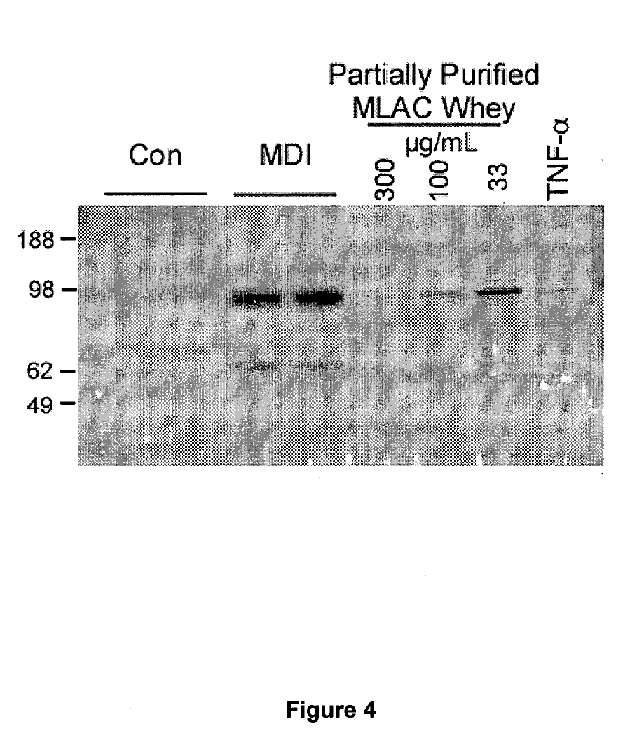

A purified MLAC Whey fraction that was shown to inhibit adipocyte differentiation and accumulation of lipid was further characterised by determining its effects on a major signalling cascade associated with adipogenesis (FIG. 4). To quantify the effect of partially purified MLAC Whey fraction on the induction of the key adipocyte transcription factor PPARγ, 3T3-L1 preadipocytes were grown and differentiated as outlined in Example 2 in 12 well microplates. As a control for inhibition of adipocyte differentiation, cells were treated with 5 ng/ml TNF-α which was added concurrently with Induction Medium. MLAC Whey fractions were also added at three concentrations concurrently with Induction Medium to test their effects on modulating differentiation of preadipocytes to mature, lipid-laden adipocytes. Assay plates were allowed to differentiate for 6 days, during which time wells were monitored by phase contrast microscopy to document the altered cellular phenotype and accumulation of refractive lipid vesicles. Medium was then removed from the assay plates, cells were washed twice with ice-cold PBS and cell lysates were prepared by extracting in 120 μl ice-cold RIPA buffer. Total protein content of the cell lysates was quantitated by BCA assay and 25 μg protein was loaded and separated on a 4-12% w/v NuPAGE Bis-Tris gel run in MES buffer. Following transfer to nitrocellulose, Western blotting was performed using a rabbit polyclonal anti-PPARγ IgG (Upstate) that recognises both PPARγ1 and PPARγ2 as well as their phosphorylated forms. Labelled immunoreactive-PPARγ bands were visualised using a chemiluminescence reagent (ECL; Amersham) and exposure to hyperfilm.

In control undifferentiated 3T3-L1 pre-adipocytes, Western blotting demonstrated the presence of very low levels of immunoreactive PPARγ. Following 6 days of induction with Methyl-3-isobutylxanthine, Dexamethasone and Insulin (MDI), differentiation to the mature adipocyte phenotype was associated with significant upregulation of PPARγ with the presence of several immunoreactive PPARγ species evident. As expected, co-incubation with TNFα largely prevented the up regulation of PPARγ expression. Induction of adipogenesis in the presence of the purified MLAC Whey fraction resulted in a dose-dependent inhibition of PPARγ up regulation, indicating that preventing transcriptional induction of this crucial adipocyte factor is part of the mechanism by which the MLAC Whey fraction blocks pre-adipocyte differentiation.

### Example 5

### Dairy-Derived Extract LAP001 is a Potent Anti-adipogenic Agent that Significantly Reduces Body Weight Gain

Compound LAP001 is a specific batch of the bioactive MLAC whey preparation described in the previous Examples. A scaled up version of the method described in Example 1 was used to generate gram quantities of LAP001 to test the efficacy of this preparation in an animal model. The LAP001 enrichment process described provides a scalable technique adaptable for large scale industrial applications. The acetic acid eluted LAP001 fraction was used for all the subsequent in vitro and in vivo studies. The bioactivity of LAP001 was confirmed in vitro and it was shown that adipocyte differentiation and lipid accumulation was inhibited over a wide concentration range (3.9 to 500 μg/ml).

An appropriate well characterised animal model utilising adult Sprague Dawley rats fed a high fat diet was used to test the efficacy of LAP001 in preventing or limiting body weight gain in vivo as well as determining its effects on a range of related metabolic parameters. The animal study comprised of three treatment groups of 10 animals per group; Vehicle, High dose LAP001 (2.5 mg/rat/day) and Low dose LAP001 (0.5 mg/rat/day). Rats were fed a high fat diet containing 23% fat from lard and canola oil for a period of 28 days. Constant subcutaneous administration was achieved via osmotic mini-pump providing continuous infusion of LAP001 and vehicle for a period of 28 days (mini-pumps were changed half way through the study at day 14). The study was designed to examine a broad range of endpoints relevant to various parameters that include: body weight prior to and throughout study; post mortem weight of organs and fat deposition; blood glucose levels at beginning and at post mortem; post mortem blood plasma insulin, leptin and adiponectin.

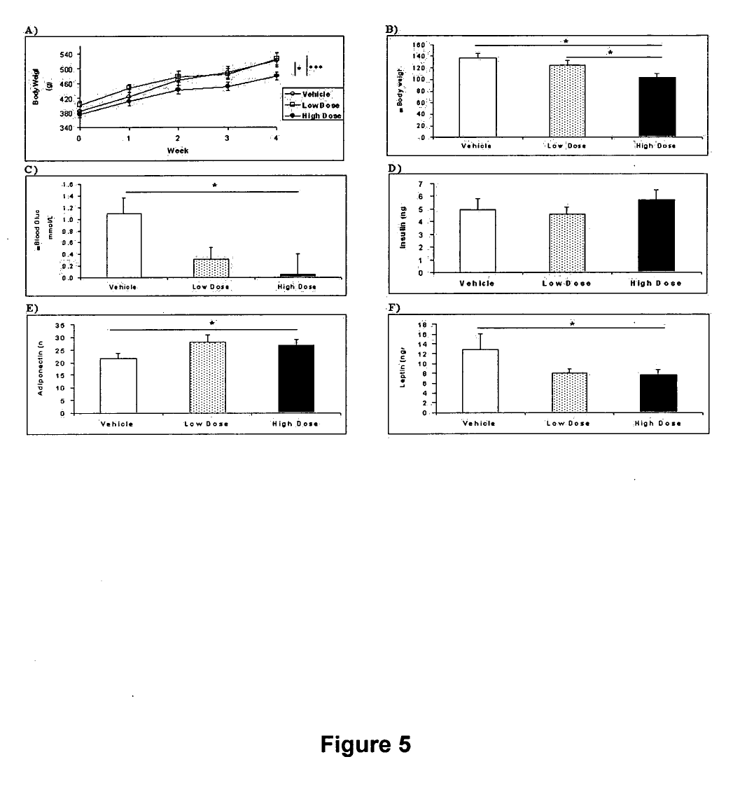

The animal study showed that LAP001 at both the high and low dose was effective at preventing several metabolic perturbations associated with a high fat diet. FIG. 5 illustrates some of the significant findings of LAP001 administration in a high fat fed animal model. All variables are expressed as mean±standard error of the mean (SEM) or mean difference±standard error of the difference (SED) (n=10). Statistical analysis of the data was performed by a multifactor repeated measure Analysis of Variance (ANOVA). *: P<0.05; ***: P<0.001.

Administration of a high fat diet led to a progressive weight gain over time in all groups, however, animals treated with LAP001 showed a dose-dependent lowering of weight compared with controls (FIG. 5A). The vehicle treated animals gained 137±8 grams while LAP001 treated animals gained significantly less weight than controls with the low and high dose groups gaining 125±7.0 grams and 103±6.5 grams respectively (P<0.05 versus vehicle for both doses) (FIG. 5B). These data clearly demonstrate the ability of LAP001 to act in vivo to prevent body weight gain in a high fat diet animal model.

Furthermore, LAP001 administration prevented the increase in blood glucose levels associated with high fat diet and weight gain in a dose-dependent manner (FIG. 5C).

Blood glucose levels increased in the vehicle group over the course of the study by 1.1±0.26 mmol/L, but not in animals treated with either low or high doses of LAP001 (0.31±0.21 mmol/L and 0.06±0.35 mmol/L respectively, P<0.05 versus vehicle treated group) (FIG. 5C). While LAP001 prevented the rise in blood glucose levels in animals fed a high fat diet, there was no difference in plasma insulin levels between groups suggesting that insulin sensitivity was maintained (FIG. 5D).

Additional parameters measured included circulating levels of the adipokines adiponectin and leptin, both of which play a role in the pathogenesis of obesity-related disorders. It is understood that low plasma levels of adiponectin and high plasma levels of leptin are commonly associated with obesity in animal models and in man. Rats treated with both doses of LAP001 had similarly increased levels of adiponectin compared to vehicle control groups (Vehicle, 21.7±1.9 ng/mL; Low LAP001, 28.1±2.8 ng/mL; High LAP001, 26.8±2.2 ng/mL, P<0.05) (FIG. 5E). Conversely, lower levels of leptin were observed for both LAP001 treated groups compared to vehicle control group (Vehicle, 12.9±3.2 ng/mL; Low LAP001, 8.1±0.8 ng/mL; High LAP001, 7.7±1.0 ng/mL, P<0.05) (FIG. 5F). These data demonstrate that in vivo administration of LAP001 has beneficial effects on major adipokines involved in metabolic consequences of weight gain and a high fat diet.

Collectively, these data illustrate that LAP001 is an effective agent that can act in vivo to ameliorate detrimental effects of a high fat diet including body weight gain and related metabolic parameters such as plasma glucose levels.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Autelitano et al, Drug Discov. Today 11(7/8):306-314, 2006
Marshall, Altern Med. Rev. 9(2):136-156, 2004
Meisel, Curr Med. Chem. 12(16):1905-1919, 2005
Meisel and Bockelmann, Antonie van Leeuwenhoek 76(1-4):207-215, 1999
Morrison and Framer, J. Nutrit. 130(12):3116S-3121S, 2000
Nicholson et al, Br. J. Pharmacol. 151:807-815, 2007
Ntambi and Kim, J. Nutrit. 130(12):3122S-3126S, 2000
Smas and Sul, Biochem. J. 309: 697-710, 1995
Student et al, J. Biol. Chem. 255(10):4745-4750, 1980
Tanaka et al, Biosci. Biotecnol. Biochem. 67(10):2254-2261, 2003
Xing et al, Endocrinology 138(7):2776-2783, 1997
Yang and Hancock, J. Chromatography A 1053: 79-88, 2004

1-22. (canceled)

23. A method for inhibiting adipogenesis or a process associated with adipogenesis in a subject, said method comprising administrating to said subject an effective amount of an adipogenesis regulator, said regulator of adipogenesis being a glycoprotein from whey or a whey fraction comprising one or more glycoproteins or a cryptein or mutein of one or more of these glycoproteins or a milk-derived fraction comprising α-casein, κ-casein and/or lactoperoxidase wherein the adipogenesis regulator is obtained by the method comprising subjecting the whey to a single multi-lectin fractionation, the lectins selected from concanavalin A, jacalin and wheat germ agglutinin or at least one other lectin substituting for concanavalin A, jacalin or wheat germ agglutinin or different combinations of lectins and then screening a fraction or isolated molecule resulting therefrom for an ability to inhibit adipogenesis in vitro wherein a fraction or molecule which inhibits adipogenesis in vitro is the regulator of adipogenesis.

24. The method of claim 23, wherein the process associated with adipogenesis is obesity.

25. The method of claim 23, wherein the process associated with adipogenesis is hypertension, diabetes or heart disease.

26. The method of claim 23, wherein the adipogenesis regulator is a glycoprotein from whey or is a whey fraction comprising one or more glycoproteins or a cryptein or mute in thereof.

27. The method of claim 23, wherein the adipogenesis regulator is a milk-derived fraction comprising α-casein, κ-casein and/or lactoperoxidase or a cryptein or mutein thereof.

28. The method of claim 23, wherein the subject is a human.

29. The method of claim 23, wherein the subject is a livestock, racing industry or companion animal.

30. The method of claim 23, wherein administration is by oral, subcutaneous, intravenous or rectal administration.

31. A method for enriching whey for glycoproteins and proteins which have adipogenesis regulatory activity and/or which control weight gain, said method comprising subjecting the whey to a single multi-lectin fractionation, the lectins selected from concanavalin A, jacalin and wheat germ aggulutinin or at least one other lectin substituting for concanavalin A, jacalin or wheat germ agglutinin or different combinations of lectins and then screening a fraction or isolated molecule resulting therefrom for an ability to inhibit adipogenesis in vitro.

32. The method of claim 31 wherein the screened fraction which inhibits adipogenesis in vitro is a glycoprotein from whey or a whey fraction comprising one or more glycoprotein or a cryptein or mutein thereof.

33. A formulation comprising a fraction or isolated molecule purified by subjecting whey to a single multi-lectin fractionation, the lectins selected from concanavalin A, jacalin and wheat germ aggulutinin or at least one other lectin substituting for concanavalin A, jacalin or wheat germ agglutinin or different combinations of lectins, wherein the fraction or isolated molecule inhibits adipogenesis in vitro.

34. The formulation of claim 33, wherein said regulator of adipogenesis is a glycoprotein from whey or a whey fraction comprising one or more glycoproteins or a cryptein or mutein of one or more of these glycoproteins or a milk derived fraction comprising α-casein, κ-casein and/or lactoperoxidase.

35. The formulation of claim 33 wherein the formulation is an oral, subcutaneous, intravenous or rectal formulation.

36. A therapeutic composition comprising the formulation of claim 33 and one or more pharmaceutically acceptable carriers, diluents and/or excipients.
